(19) United States
(12) Patent Application Publication
(10) Pub. No.: US 2013/0150622 A1
(43) Pub. Date: Jun. 13, 2013

(54) STEREOSELECTIVE SYNTHESIS OF TAPENTADOL AND ITS SALTS

(71) Applicant: Boehringer Ingelheim International GmbH, Ingelheim am Rhein (DE)

(72) Inventors: Daniel Robert FANDRICK, Danbury, CT (US); Sonia RODRIGUEZ, New Milford, CT (US); Bing-Shiou YANG, Southbury, CT (US)

(73) Assignee: BOEHRINGER INGELHEIM INTERNATIONAL GMBH, Ingelheim am Rhein (DE)

(21) Appl. No.: 13/709,571

(22) Filed: Dec. 10, 2012

Related U.S. Application Data
(60) Provisional application No. 61/569,437, filed on Dec. 12, 2011.

(51) Int. Cl. C07C 213/02; C07C 215/54

(52) U.S. Cl. CPC C07C 213/02; C07C 215/54
USPC 564/374

(57) ABSTRACT

A process for the synthesis of a salt of tapentadol.

### FIELD OF THE INVENTION

The present invention relates to the stereoselective synthesis of tapentadol and its salts.

### BACKGROUND OF THE INVENTION

Tapentadol, (−)-(1R,2R)-3-(3-dimethylamino-1-ethyl-2-methylpropyl)phenol, is a synthetic, centrally acting analgesic which is effective in the treatment of moderate to severe, acute, or chronic pain and has the following structure:

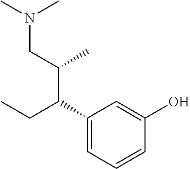

Tapentadol is described in U.S. Pat. No. 6,248,737. Tapentadol exhibits a dual mechanism of action, on the one hand as a n-opioid receptor agonist and on the other as a noradrenaline transporter inhibitor. Tapentadol can be used in the form of its free base or as a salt or solvate. The production of the free base is known, for example, from EP-A 693 475. Suitable pharmaceutically acceptable salts include salts of inorganic acids, such as hydrochloric acid, hydrobromic acid, and sulfuric acid, and salts of organic acids, such as methanesulfonic acid, fumaric acid, maleic acid, acetic acid, oxalic acid, succinic acid, malic acid, tartaric acid, mandelic acid, lactic acid, citric acid, glutaminic acid, acetylsalicylic acid, nicotinic acid, aminobenzoic acid, α-lipoic acid, hippuric acid, and aspartic acid.

Various processes for the preparation of tapentadol, its enantiomers, and related compounds, and their pharmaceutically acceptable salts are disclosed in U.S. Pat. No. 6,248,737 and PCT Publication Nos. WO 2004/108658, WO 2005/000788, WO 2008/012046, WO 2008/012047 and WO 2008/012283.

It is well known in the art that enantiomers of a particular compound can have different biological properties including efficacy, toxicity, and pharmacokinetic properties. Thus, it is often desirable to administer one enantiomer of a racemic therapeutic compound.

The synthetic methods disclosed in the patent applications cited above describe the synthesis of racemic products. Separation of enantiomers was accomplished by chiral HPLC and may be accomplished by other conventional ways of separating enantiomers. Chiral HPLC and other enantiomer separation methods, however, are generally unsuitable for large-scale preparation of a single enantiomer. Thus, a stereoselective synthesis for preparation of these compounds would be highly desirable.

### SUMMARY OF THE INVENTION

The instant invention is directed to a process for synthesis of a salt of tapentadol, the process comprising:
(a) reacting (E)-3-(3-hydroxyphenyl)acrylic acid in a suitable solvent with benzyl bromide to obtain (E)-3-(3-(benzyloxy)phenyl)acrylic acid

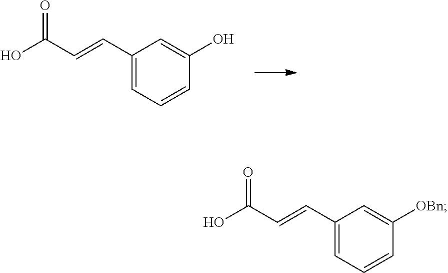 [SMILES: O=C(O)/C=C/c1cccc(O)c1 ; O=C(O)/C=C/c1cccc(OCc2ccccc2)c1]

(b) reducing (E)-3-(3-(benzyloxy)phenyl)acrylic acid in a suitable solvent to obtain (E)-3-(3-(benzyloxy)phenyl)prop-2-en-1-ol

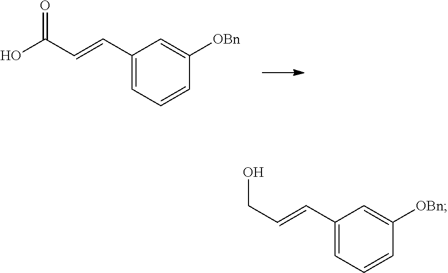 [SMILES: O=C(O)/C=C/c1cccc(OCc2ccccc2)c1 ; OC/C=C/c1cccc(OCc2ccccc2)c1]

(c) reacting (E)-3-(3-(benzyloxy)phenyl)prop-2-en-1-ol with allyl bromide in a suitable solvent to obtain (E)-1-(3-(allyloxy)prop-1-enyl)-3-(benzyloxy)benzene

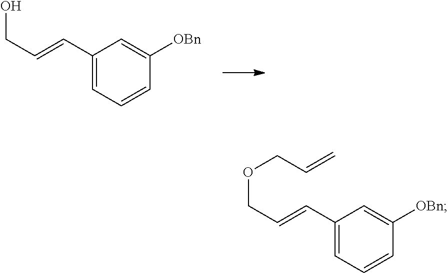 [SMILES: OC/C=C/c1cccc(OCc2ccccc2)c1 ; C=CCOC/C=C/c1cccc(OCc2ccccc2)c1]

(d) isomerizing (E)-1-(3-(allyloxy)prop-1-enyl)-3-(benzyloxy)benzene with an organometallic catalyst in a suitable solvent to obtain 1-(benzyloxy)-3-((E)-3-((E)-prop-1-enyloxy)prop-1-enyl)benzene

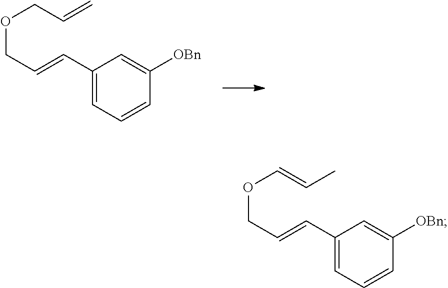 [SMILES: C=CCOC/C=C/c1cccc(OCc2ccccc2)c1 ; C/C=C/OC/C=C/c1cccc(OCc2ccccc2)c1]

(e) reacting 1-(benzyloxy)-3-((E)-3-((E)-prop-1-enyloxy)prop-1-enyl)benzene with 4-(dimethylamino)-N-((1R,2S)-2-hydroxy-2,3-dihydro-1H-inden-1-yl)picolinamide in the presence of a suitable organometallic catalyst to obtain (2R,3R)-3-(3-(benzyloxy)phenyl)-2-methylpent-4-enal

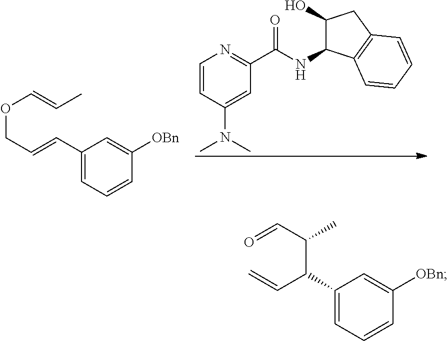

(f) reducing (2R,3R)-3-(3-(benzyloxy)phenyl)-2-methylpent-4-enal in a suitable solvent with a suitable reducing agent to obtain (2R,3R)-3-(3-(benzyloxy)phenyl)-N,N,2-trimethylpent-4-en-1-amine

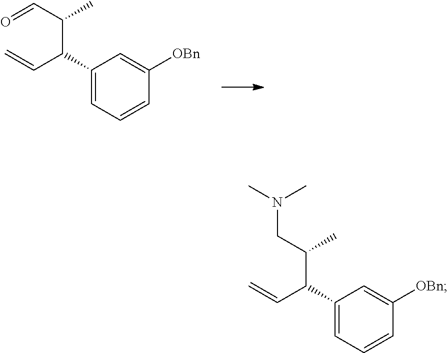

(g) reacting (2R,3R)-3-(3-(benzyloxy)phenyl)-N,N,2-trimethylpent-4-en-1-amine with a suitable acid HX to obtain a salt of (2R,3R)-3-(3-(benzyloxy)phenyl)-N,N,2-trimethylpent-4-en-1-amine

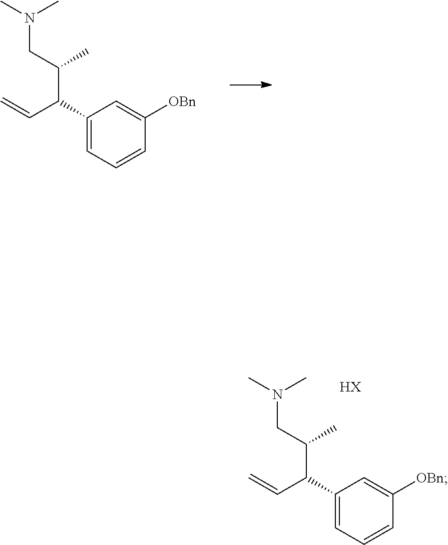

and
(h) hydrogenating the salt of (2R,3R)-3-(3-(benzyloxy)phenyl)-N,N,2-trimethylpent-4-en-1-amine to obtain the salt of tapentadol

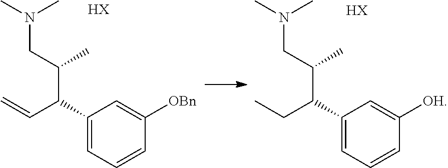

In the above, the HX designation for the suitable acid is used for convenience only, and may represent any suitable acid, such as hydrochloric acid, as well as diprotic or polyprotic acids such as sulfuric acid or phosphoric acid.

In an aspect of the invention, the suitable solvent of steps (a) to (g) are each organic solvents, preferably anhydrous organic solvents.

In an aspect of the invention, the suitable solvent of step (a) is anhydrous dimethylformamide (DMF), anhydrous dimethylsulfoxide (DMSO), anhydrous dimethyl acetamide (DMAc), anhydrous ethanol, anhydrous methanol, anhydrous n-propanol, anhydrous 2-butanol, anhydrous 1-butanol, anhydrous tetrahydrofuran (THF), anhydrous 2-methyltetrahydrofuran (2-MeTHF), anhydrous dioxane, anhydrous toluene, anhydrous ethyl acetate, anhydrous isopropyl acetate, or a mixture thereof, preferably anhydrous DMF, anhydrous DMSO, anhydrous DMAc, or anhydrous ethanol.

In an aspect of the invention, the suitable solvent of step (b) is anhydrous 2-MeTHF, anhydrous THF, anhydrous toluene, anhydrous dioxane, anhydrous methyl tert-buyl ether (MTBE), anhydrous cyclopentyl methyl ether, or anhydrous diethyl ether, preferably anhydrous 2-MeTHF or anhydrous THF.

In an aspect of the invention, step (b) is accomplished using a suitable reducing agent, the reducing agent preferably selected from lithium borohydride, sodium borohydride, lithium aluminum hydride, disobutyl aluminum hydride, or RedAl, most preferably lithium borohydride or sodium borohydride.

In an aspect of the invention, the suitable solvent of step (c) is THF, 2-MeTHF, toluene, dioxane, MTBE, cyclopentyl methyl ether, diethyl ether, or a mixture thereof, preferably THF or 2-MeTHF.

In another aspect of the invention, the organometallic catalyst of step (d) is [Ir(COD)Cl]₂ or ]Ir(COE)₂Cl]₂, preferably [Ir(COD)Cl]₂.

In still another aspect of the invention, the suitable solvent of step (d) is dichloromethane, THF, toluene, dioxane, MTBE, cyclopentyl methyl ether, diethyl ether, acetone, or a mixture thereof, preferably dichloromethane or toluene.

In another aspect of the invention, the 4-(dimethylamino)-N-((1R,2S)-2-hydroxy-2,3-dihydro-1H-inden-1-yl)picolinamide is obtained by reacting 4-(dimethylamino)picolinic acid with (1R,2S)-1-amino-2,3-dihydro-1H-inden-2-ol in a suitable solvent, preferably anhydrous DMF

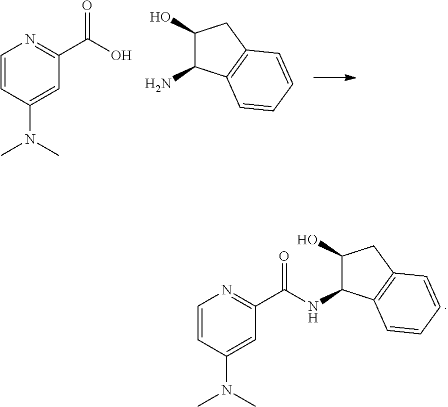 [SMILES: CN(C)c1ccnc(C(=O)O)c1 ; N[C@@H]1c2ccccc2C[C@@H]1O ; CN(C)c1ccnc(C(=O)N[C@@H]2c3ccccc3C[C@@H]2O)c1]

In another aspect of the invention, the organometallic catalyst of step (e) is [CpRu(MeCN)₃]PF₆ or [Cp*Ru(MeCN)₃]PF₆, preferably [CpRu(MeCN)₃]PF₆.

In an aspect of the invention, the suitable solvent of step (e) is anhydrous THF, anhydrous 2-MeTHF, anhydrous toluene, anhydrous dioxane, anhydrous MTBE, anhydrous cyclopentyl methyl ether, anhydrous diethyl ether, or a mixture thereof, preferably anhydrous THF or anhydrous 2-MeTHF.

In an aspect of the invention, the suitable solvent of step (f) is THF, 2-MeTHF, toluene, dioxane, MTBE, cyclopentyl methyl ether, diethyl ether, dichloromethane, dichloroethane, or a mixture thereof, preferably THF or 2-MeTHF.

In an aspect of the invention, the suitable solvent of step (g) is MTBE, cyclopentyl methyl ether, diethyl ether, acetonitrile, or a mixture thereof, preferably MTBE, cyclopentyl methyl ether, or diethyl ether.

In an aspect of the invention, the suitable acid HX of step (g) is hydrochloric acid, hydrobromic acid, sulfuric acid, methanesulfonic acid, fumaric acid, maleic acid, acetic acid, oxalic acid, succinic acid, malic acid, tartaric acid, mandelic acid, lactic acid, citric acid, glutaminic acid, acetylsalicylic acid, nicotinic acid, aminobenzoic acid, α-lipoic acid, hippuric acid, or aspartic acid, preferably hydrochloric acid.

In another aspect of the invention, the salt of step (g) is obtained by seeding with crystals of the salt of (2R,3R)-3-(3-(benzyloxy)phenyl)-N,N,2-trimethylpent-4-en-1-amine. Such seeds were obtained the seeds by carefully running the desired crystallization to afford some crystalline solids and these solids were then used to crystallize the bulk material.

In another aspect of the invention, the hydrogenation of step (h) is accomplished with a hydrogenation catalyst, preferably palladium catalyst on carbon.

In another aspect of the invention, the salt of tapentadol is converted to tapentadol by reaction with a base.

The invention also comprises the key intermediate in the process, a salt of (2R,3R)-3-(3-(benzyloxy)phenyl)-N,N,2-trimethylpent-4-en-1-amine:

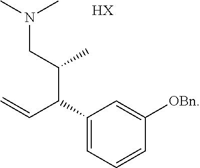

In another aspect, the invention also comprises the key intermediate in the process, the salt of (2R,3R)-3-(3-(benzyloxy)phenyl)-N,N,2-trimethylpent-4-en-1-amine, preferably the chloride salt of (2R,3R)-3-(3-(benzyloxy)phenyl)-N,N,2-trimethylpent-4-en-1-amine:

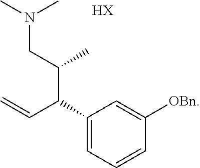

In other aspect, the invention comprises the use of this key intermediate, crystallization of this key intermediate and polymorphs thereof, and purfication of this key intermediate and polymorphs thereof.

### DETAILED DESCRIPTION OF THE INVENTION

Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### EXPERIMENTAL EXAMPLES

The invention provides processes for making tapentadol and its salts and derivatives. Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Experimental Examples section. Typically, reaction progress may be monitored by high performance liquid chromatography (HPLC) or thin layer chromatography (TLC), if desired, and intermediates and products may be purified by chromatography on silica gel by recrystallization and/or distillation. Normal room temperature means about 20° C., although the exact tempeature was not measured, since it was not considered significant in the experiment below.

### SYNTHETIC EXAMPLE

The following reaction Scheme 1 is representative of the process that indicates the various steps, reagents, and yields.

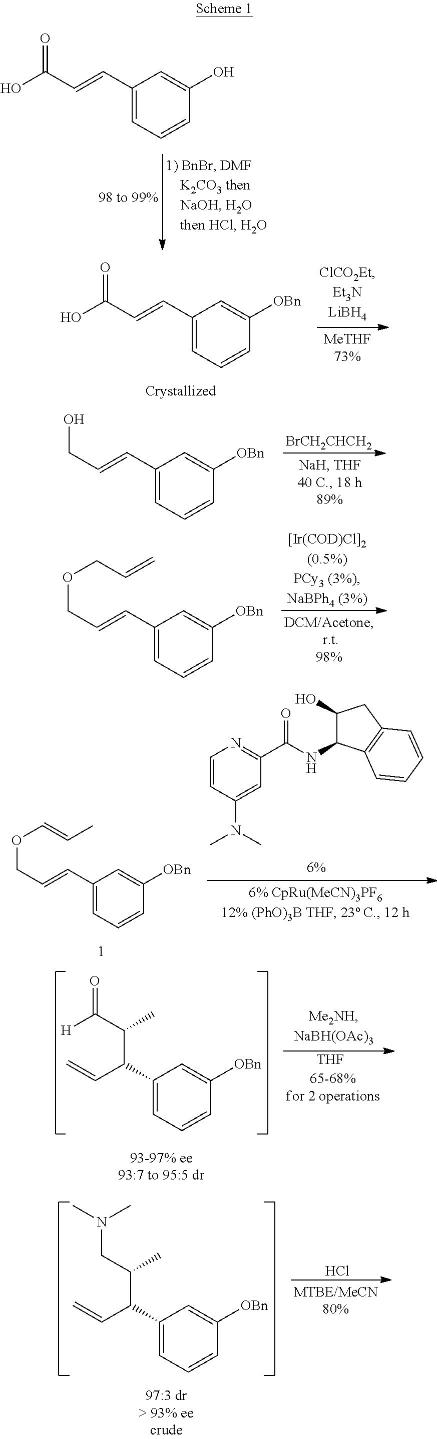

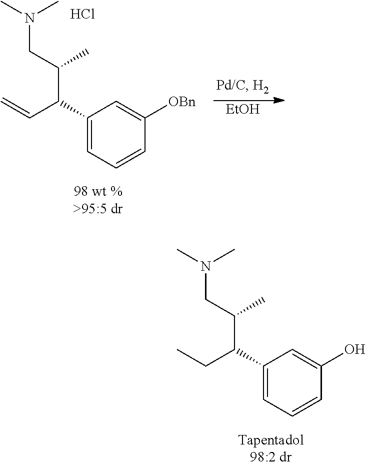

The various steps of the process of Scheme 1 are outlined in more detail below.

### (E)-3-(3-(benzyloxy)phenyl)acrylic acid

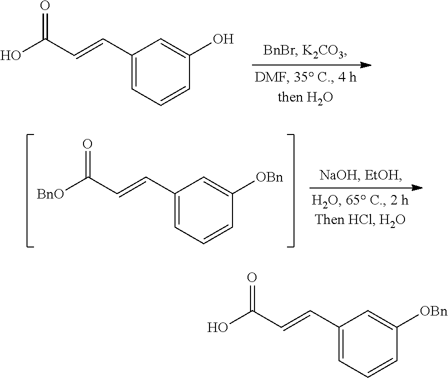

(E)-3-(3-hydroxyphenyl)acrylic acid (100 g, 606 mmol) was charged to a 2 L jacketed reactor. The reactor was flushed with nitrogen, charged with 800 mL of anhydrous DMF, and agitation was started. Solid potassium carbonate (250 g, 1.81 mol) was charged to the reaction at a rate to maintain the batch temperature between 20° C.-30° C. Benzyl bromide (160 mL, 1.35 mol) was charged to the reaction, and the reaction was agitated at 35° C. for 4 hours and until conversion to the bis-benzyl adduct was >95%. The reaction was cooled to 22° C. and 1200 mL of water was charged to the reaction. The reaction was cooled to 10° C., at which point, 800 mL of water was charged to the reaction over 100 minutes. The resulting mixture was agitated for 1 hour. The solids were collected by filtration and washed with water. The wet solids were charged back to the 2 L jacketed reactor followed by 650 mL of ethanol. The solution was warmed to 55° C., at which point, aqueous sodium hydroxide (2M, 790 mL) was charged to the reaction. The reaction was agitated at 55° C. for 45 minutes and until saponification was >98%. The reaction was cooled to normal room temperature. 600 mL of aqueous HCl (3M) was charged to the reaction at 20° C. dropwise over 70 minutes to crystallize the desired product. The resulting mixture was agitated at normal room temperature for 1 to 18 hours. The solids were collected by filtration and washed with water followed by heptane. The solids were dried in a vacuum oven with a nitrogen stream at 50° C. for 18 hours to provide the desired product (E)-3-(3-(benzyloxy)phenyl)acrylic acid as a white solid (151.24 g, 100 wt %, 98% yield). ¹H NMR (400 MHz, CDCl₃) δ 7.75 (d, J=15.84 Hz, 1H), 7.47-7.28 (m, 6H), 7.19-7.14 (m, 2H), 7.03 (d, J=8.1 Hz, 1H), 6.43 (d, J=16.1 Hz, 1H), 5.10 (s, 2H).

### (E)-3-(3-(benzyloxy)phenyl)prop-2-en-1-ol

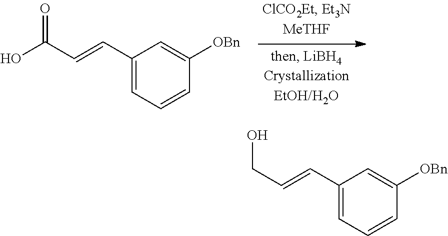 [SMILES: O=C(O)/C=C/c1cccc(OCc2ccccc2)c1 ; OC/C=C/c1cccc(OCc2ccccc2)c1]

(E)-3-(3-(benzyloxy)phenyl)acrylic acid (151 g, 594 mmol) was charged to a 2 L jacketed reactor. The reactor was flushed with nitrogen and anhydrous 2-methyltetrahydrofuran (MeTHF, 1.2 L) was charged to the reactor. Agitation was started, and triethylamine (90.0 mL, 646 mmol) was charged to the reactor at a rate to maintain the batch temperature between 10° C. and 20° C. Ethyl chloroformate (60.0 mL, 627 mmol) was charged to the reactor at a rate to maintain the batch temperature between 10° C. and 20° C. The reaction was agitated at 10° C. and 20° C. for 1 hour. Lithium borohydride solution (2.0 M in THF, 290 g, 647 mmol) was charged to the reactor at a rate to maintain the batch temperature between 10° C. and 20° C. The reaction was agitated at 10° C. and 20° C. for 2 hours. 140 mL of water was slowly charged to the reactor at a rate to maintain the batch temperature between 10° C. and 20° C. Aqueous sodium hydroxide (2 M, 480 mL, was slowly charged to the reactor over 30 minutes with a batch temperature between 10° C. and 20° C. 480 mL of water was charged to the reactor. The layers were separated, and the organic portion was washed with 300 mL of water. The organic portion was concentrated in vacuo to 400 mL. 500 mL of ethanol was charged to the reactor and the solution was concentrated in vacuo to 400 mL. This chase was repeated. 150 mL of ethanol was charged to the batch, and the batch temperature was adjusted to 0° C. The solution was seeded to induce crystallization, and the resulting mixture was diluted slowly with 400 mL of water at −5° C. to 0° C. The resulting mixture was agitated for 1 hour at 0° C. The solids were collected by filtration and washed with water. The solids were dried in a vacuum oven with a nitrogen stream at 20° C. to afford the desired (E)-3-(3-(benzyloxy)phenyl)prop-2-en-1-ol as a white solid and the semi-ethanol solvate (114 g, 91 wt %, 4.3 wt % ethanol, 73% yield). ¹H NMR (400 MHz, CDCl₃) δ 7.46-7.28 (m, 5H), 7.22 (dd, J=8.0, 8.0 Hz, 1H), 7.02-6.96 (m, 2H), 6.86 (d, J=8.0 Hz, 1H), 6.56 (d, J=15.8 Hz, 1H), 6.33 (ddd, J=15.8, 5.6, 5.6 Hz, 1H), 5.05 (s, 2H), 4.29 (d, J=5.6 Hz, 2H).

### (E)-1-(3-(allyloxy)prop-1-enyl)-3-(benzyloxy)benzene

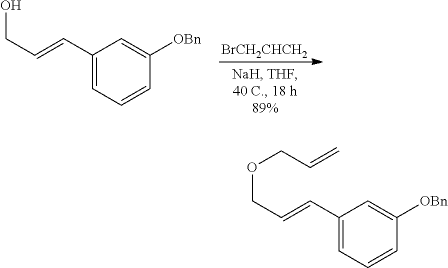

Sodium hydride (60%, 4.99 g, 125 mmol) was charged to a suitable reactor, and the system was flushed with nitrogen. 100 mL of anhydrous THF was charged to the reactor and agitation was started. (E)-3-(3-(benzyloxy)phenyl)prop-2-en-1-ol (21.3 g, 83.2 mmol) as a solution in 100 mL of THF was charged to the reactor at a rate to maintain the batch temperature between 20° C. and 30° C. Allyl bromide (10.8 mL, 125 mmol) was charged to the reactor. The reaction was agitated at 40° C. for 18 hours. The reaction was diluted with 400 mL of heptane and slowly quenched with 300 mL of water. The layers were separated and the organic portion was washed with 300 mL of water. The organic portion was purified by silica gel chromatography (4% IpAc in heptane) to provide the intended product (E)-1-(3-(allyloxy)prop-1-enyl)-3-(benzyloxy)benzene as a colorless and clear oil (20.7 g, 89% yield). ¹H NMR (400 MHz, CDCl₃) δ 7.46-7.30 (m, 5H), 7.22 (t, J=7.7 Hz, 1H), 7.09-6.95 (m, 2H), 6.86 (d, J=8.1 Hz, 1H), 6.58 (d, J=15.7 Hz, 1H), 6.28 (ddd, J=6.26, 6.0, 6.0 Hz, 1H), 5.95 (ddd, J=17.3 Hz, 10.6 Hz, 5.71 Hz, 1H), 5.31 (d, J=17.3 Hz, 1H), 5.21 (d, J=10.5 Hz, 1H), 5.06 (s, 2H), 4.15 (d, J=5.96 Hz, 1H), 4.03 (d, J=5.3 Hz, 1H).

### 1-(benzyloxy)-3-((E)-3-((E)-prop-1-enyloxy)prop-1-enyl)benzene

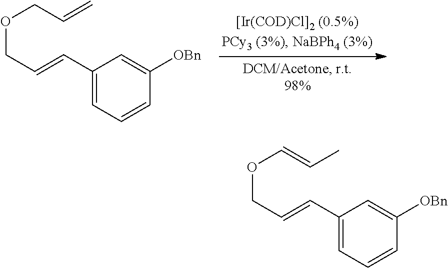 [SMILES: C=CCOC/C=C/c1cccc(OCc2ccccc2)c1 ; C/C=C/OC/C=C/c1cccc(OCc2ccccc2)c1]

[Ir(COD)Cl]₂ (239 mg, 0.356 mmol), tricyclohexylphosphine (600 mg, 2.13 mmol), and sodium tetraphenylborate (732 mg, 2.13 mmol) were charged under nitrogen to a suitable reactor previously flushed with nitrogen. Degassed dichloromethane (84 mL) followed by degassed acetone (4 mL) were charged to the reaction and agitation was started to afford a homogeneous solution. (E)-1-(3-(allyloxy)prop-1-enyl)-3-(benzyloxy)benzene (20 g, 71 mmol) as an oil was charged to the reactor at normal room temperature and the reaction was agitated at this temperature for 14 hours. The reaction was diluted with 200 mL of MTBE and passed through a silica plug. The filtrate was concentrated to an oil. The oil was passed through a silica plug with 5% MTBE in hexanes. The filtrate was concentrated to an oil which solidified upon standing into the desired product 1-(benzyloxy)-3-((E)-3-((E)-prop-1-enyloxy)prop-1-enyl)benzene as a yellow solid (21.05 g, 91 wt %, 96% yield). ¹H NMR (400 MHz, C₆D₆) δ 7.26 (d, J=7.56 Hz, 2H), 7.18-7.12 (m, 2H), 7.11-7.00 (m, 3H), 6.89 (d, J=7.5 Hz, 1H), 6.77 (dd, J=8.2, 2,1 Hz, 1H), 6.50 (d, J=15.9 Hz, 1H), 6.26 (d, J=12.5 Hz, 1H), 6.16 (ddd, J=15.9, 5.6 Hz, 1H), 4.84 (ddd, J=12.7, 6.3, 6.3 Hz, 1H), 4.68 (s, 2H), 4.07 (d, J=5.61 Hz, 2H), 1.48 (d, J=6.70 Hz, 3H).

### 4-(Dimethylamino)-N-((1R,2S)-2-hydroxy-2,3-dihydro-1H-inden-1-yl)picolinamide

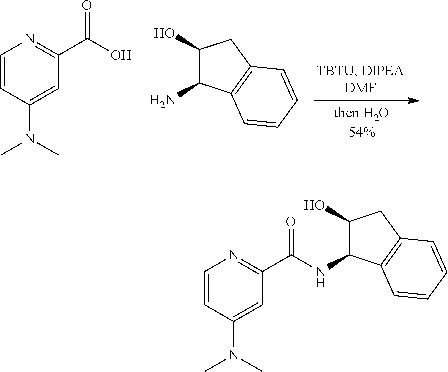 [SMILES: CN(C)c1ccnc(C(=O)O)c1 ; N[C@@H]1c2ccccc2C[C@@H]1O ; CN(C)c1ccnc(C(=O)N[C@@H]2c3ccccc3C[C@@H]2O)c1]

Anhydrous DMF (25 mL) was charged to a dried reactor charged with 4-(dimethylamino)picolinic acid (1.80 g, 12.1 mmol) and O-(benzotriazol-1-yl)-N,N,N′,N′-tetramethyluronium tetrafluoroborate (4.26 g, 13 mol) under nitrogen at normal room temperature. Agitation was started and isopropyl ethylamine (2.31 mL, 13 mmol) was charged to the slurry. After agitating the slurry for 30 minutes, a homogeneous solution was obtained. (1R,2S)-1-amino-2,3-dihydro-1H-inden-2-ol (2.05 g) was charged to the reactor as a solid followed by diisopropyl ethylamine (2.31 mL, 13 mmol). The reaction was agitated at normal room temperature under argon for 18 hours. 40 mL of water was charged to the reactor dropwise over 1 hour. The resulting slurry was agitated at normal room temperature for 1 hour. The solids were collected by filtration and washed with water followed by heptane. The solids were dried in a vacuum oven with a nitrogen stream at 70° C. for 2 days to provide the intended product 4-(dimethylamino)-N-((1R,2S)-2-hydroxy-2,3-dihydro-1H-inden-1-yl)picolinamide as an off white solid (2.74 g, 76% yield). ¹H NMR (400 MHz, DMSO-D₆) δ 8.77 (d, J=8.58 Hz, 1H), 8.15 (d, J=5.8 9 Hz, 1H), 7.37 (d, J=2.8 Hz, 1H), 7.29-7.13 (m, 4H), 6.76 (dd, J=5.9, 2.8 Hz, 1H), 5.45 (d, J=4.7 Hz, 1H), 5.34 (dd, J=8.7, 5.1 Hz, 1H), 4.51 (dd, J=4.71, 4.71, 4.71 Hz, 1H), 3.14 (dd, J=16.2, 4.8 Hz, 1H), 3.05 (s, 6H), 2.86 (d, J=16.2 Hz, 1H).

### (2R,3R)-3-(3-(benzyloxy)phenyl)-N,N,2-trimethylpent-4-en-1-amine HCl

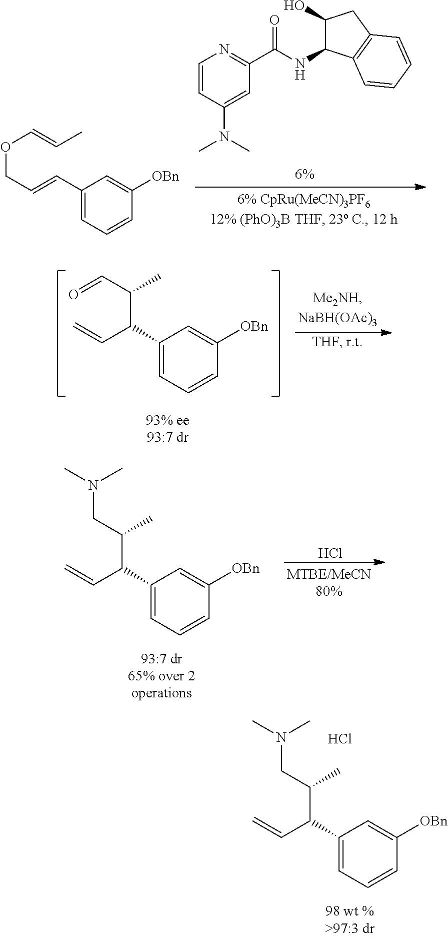

Under nitrogen, [CpRu(MeCN)₃]PF₆ (1.45 g, 3.3 mmol) and 4-(dimethylamino)-N-((1R,2S)-2-hydroxy-2,3-dihydro-1H-inden-1-yl)picolinamide (1.04 g, 3.5 mmol) were charged to a dried 25 mL round bottom flask. This ruthenium catalyst has been previously utilized in an asymmetric Claisen reaction by M. E. Geherty, R. D. Dura, and S. G. Nelson, J. Am. Chem. Soc. 2010, 132, 11875-11877. Degassed THF (13 mL) was charged to the flask under nitrogen at normal room temperature and the mixture was agitated for 30 minutes to afford the catalyst solution as a homogeneous solution. Under nitrogen, 1-(benzyloxy)-3-((E)-3-((E)-prop-1-enyloxy)prop-1-enyl)benzene (17.0 g, 91.6 wt %, 55.5 mmol) and triphenyl borate (1.93 g, 6.67 mmol) were charged to a 250 mL dried round bottom flask.

Degassed THF (150 mL) was charged to the 250 mL flask under nitrogen at normal room temperature. The mixture was agitated for 20 minutes to afford a homogeneous solution. The catalyst solution was charged to the 250 mL round bottom flask reaction at normal room temperature. The reaction was agitated at 20° C. for 18 hours to afford >98 A % conversion to (2R,3R)-3-(3-(benzyloxy)phenyl)-2-methylpent-4-enal by HPLC at 205 nm. Crude NMR in C₆D₆ showed 93:7 dr, and chiral HPLC of the alcohol derivative prepared by reducing an aliquot reduced by sodium borohyride, showed 93% ee.

The reaction was charged by cannula under nitrogen at normal room temperature to an agitating suspension of sodium triacetoxyborohydride (35.3 g, 167 mmol) and dimethyl amine (2M in THF, 83.3 mL, 167 mmol) in 340 mL of anhydrous THF. The reaction was agitated at normal room temperature for 1 hour; at which point, HPLC (205 nm) showed complete reductive amination. The reaction was slowly quenched by the addition of aqueous sodium hydroxide (2M, 100 mL) followed by 100 mL of water. The mixture was agitated for 40 minutes. The reaction was diluted with 300 mL of MTBE and 100 mL of water. The layers were separated, and the organic portion was washed twice with 200 mL portions of water. The organic portion was concentrated to an oil. The oil was dissolved in 100 mL of MTBE and passed through a silica plug. The filtrate was concentrated to provide crude (2R,3R)-3-(3-(benzyloxy)phenyl)-N,N,2-trimethylpent-4-en-1-amine as an oil in 59.63 wt % (18.62 g, 59.63 wt %, 65% yield) and 93:7 dr.

Crude (2R,3R)-3-(3-(benzyloxy)phenyl)-N,N,2-trimethylpent-4-en-1-amine (5.13 g, 59.63 wt %) was charged to a suitable reactor followed by acetonitrile (15.51 g). Agitation was started and concentrated hydrochloric acid (37.3 wt %, 1.38 g) was charged to the reactor. The solution was concentrated to remove 11.8 g of distillate. Acetonitrile (20.1 g) was charged to the reactor and the solution was concentrated to remove 20.2 g of distillate. MTBE (6.12 g) was charged to the solution at 40° C. The solution at 40° C. was seeded with the (2R,3R)-3-(3-(benzyloxy)phenyl)-N,N,2-trimethylpent-4-en-1-amine HCl (31 mg). The resulting slurry was agitated at 40° C. for 70 minutes. MTBE (3.1 g) was charged over 10 minutes, and the mixture was agitated for 1 hour. MTBE (3.1 g) was charged to the mixture over 10 minutes and the mixture was agitated for 30 minutes. The mixture was cooled to 20° C. over 1 hour. The solids were collected by filtration and washed with MTBE. The solids were dried in a vacuum oven with a nitrogen stream at 60° C. to afford the desired product (2R,3R)-3-(3-(benzyloxy)phenyl)-N,N,2-trimethylpent-4-en-1-amine HCl as an off white solid in >97:3 diastereoselectivity (2.71 g, 95.1 wt %, 78% yield). ¹H NMR (500 MHz, DMSO-D₆) δ 10.2 (bs, 1H), 7.48-7.43 (m, 2H), 7.39 (dd, J=7.5, 7.5 Hz, 2H), 7.33 (dd, J=7.5, 7.5 Hz, 1H), 7.25 (dd, J=7.9, 7.9 Hz, 1H), 6.96 (s, 1H), 6.91-6.84 (m, 2H), 6.01 (ddd, J=17.1, 9.8, 9.8 Hz, 1H), 5.19-5.01 (m, 4H), 3.24 (dd, J=8.2, 8.2 Hz, 1H), 2.83 (dd, J=10, 10 Hz, 1h), 2.78-2.65 (m, 1H), 2.69 (s, 3H), 2.61 (s, 3H), 2.31-2.35 (m, 1H), 1.02 (d, J=6.6 Hz, 3H).

### Tapentadol HCl

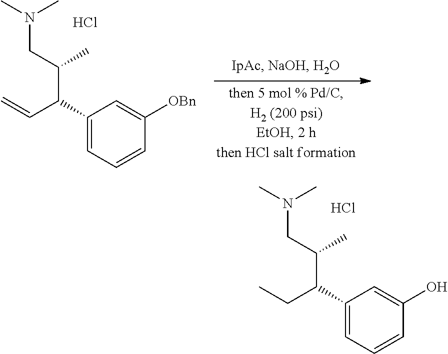

(2R,3R)-3-(3-(benzyloxy)phenyl)-N,N,2-trimethylpent-4-en-1-amine HCl (3.86 g, 11.1 mmol) was suspended in IpAc (70 mL) and treated with 60 mL of water followed by aqueous sodium hydroxide (2M, 11.1 mL). The mixture was agitated for 15 minutes, and the layers were separated. The organic portion was washed with aqueous sodium hydroxide (30 mL+2 mL 2M NaOH). The organic portion was concentrated to an oil. The oil was charged to a 70 mL hydrogenation vessel followed by 10 wt % Pd/C (10 wt % wet, 593 mg, 0.56 mmol) followed by 40 mL of ethanol. The mixture was subjected to hydrogenation at 200 psi and 20° C. for 2 hours. The reaction mixture was passed through a CELITE® filter aid plug and concentrated to an oil. The oil was dissolved in 50 mL of ethanol and treated with an ethanol HCl solution (ethanol 15 mL+3 mL concentrated aqueous HCl). The solution was concentrated to an oil, which solidified upon standing. The mixture was suspended in 100 mL of isopropyl acetate and agitated for 1 hour. The solids were collected by filtration to provide tapentadol HCl in 96.7 wt % (2.77 g, 96.7 wt %, 93% yield). ¹H NMR (D₂O, 500 MHz) δ 7.14 (dd, J=8.2, 8.2 Hz, 1H), 6.69 (d, J=8.35 Hz, 1H), 6.67 (d, 8.1 Hz, 1H), 6.62 (bs, 1H), 2.78-2.64 (m, 2H), 2.63 (s, 3H), 2.58 (s, 3H), 2.23-2.16 (m 1H), 2.09-1.99 (m, 1H), 1.77-1.66 (m, 1H), 1.50-1.38 (m, 1H), 0.94 (d, J=6.5 Hz, 3H), 0.54 (dd, J=7.4, 7.4 Hz, 3H). This ¹H NMR corresponds to K. Ravikumar, et al., Acta Crystallographica Section C, 2011, C67, o71-o76. The optical rotation for the crude HCl salt: [α]_{D}=−21.1 (c=0.29 in MeOH) corresponds to Bhirud, et al., WO 2011/080756; [α]_{D}=−27.6 (c=0.97 in MeOH).

### Additional Experimental Procedure

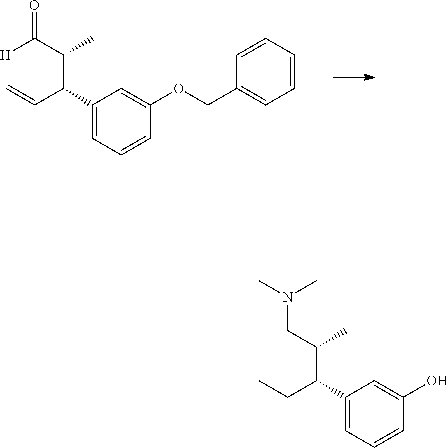 [SMILES: C=C[C@@H](c1cccc(OCc2ccccc2)c1)[C@@H](C)C=O ; CC[C@@H](c1cccc(O)c1)[C@@H](C)CN(C)C]

(2R,3R)-3-(3-(benzyloxy)phenyl)-2-methylpent-4-enal (250 mg, 0.89 mmol) was charged to a hydrogenation vessel followed by 10 wt % Pd/C (47.45 mg, 0.05 mmol) followed by methanol (4.5 mL) and dimethylamine (2M in THF, 0.54 mL, 1.07 mmol). The mixture was subjected to hydrogenation at 200 psi and 25° C. for 20 hours. The reaction mixture was passed through a CELITE® filter aid plug and concentrated to an oil (yield: 0.19 g crude tapentadol).

We claim:

1. A process for synthesis of a salt of tapentadol, the process comprising:
(a) reacting (E)-3-(3-hydroxyphenyl)acrylic acid in a suitable solvent with benzyl bromide to obtain (E)-3-(3-(benzyloxy)phenyl)acrylic acid 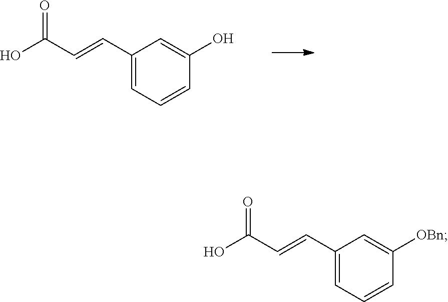 [SMILES: O=C(O)/C=C/c1cccc(O)c1 ; O=C(O)/C=C/c1cccc(OCc2ccccc2)c1]
(b) reducing (E)-3-(3-(benzyloxy)phenyl)acrylic acid in a suitable solvent to obtain (E)-3-(3-(benzyloxy)phenyl)prop-2-en-1-ol 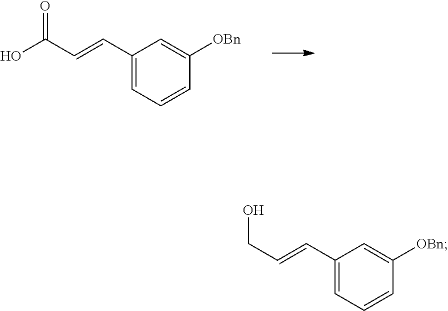 [SMILES: O=C(O)/C=C/c1cccc(OCc2ccccc2)c1 ; OC/C=C/c1cccc(OCc2ccccc2)c1]
(c) reacting (E)-3-(3-(benzyloxy)phenyl)prop-2-en-1-ol with allyl bromide in a suitable solvent to obtain (E)-1-(3-(allyloxy)prop-1-enyl)-3-(benzyloxy)benzene 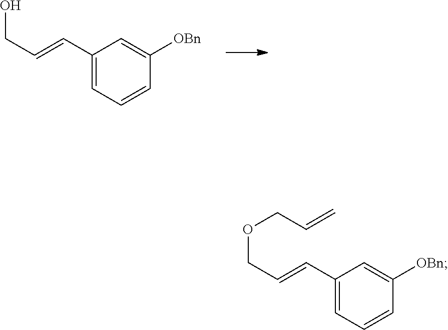 [SMILES: OC/C=C/c1cccc(OCc2ccccc2)c1 ; C=CCOC/C=C/c1cccc(OCc2ccccc2)c1]
(d) isomerizing (E)-1-(3-(allyloxy)prop-1-enyl)-3-(benzyloxy)benzene with an organometallic catalyst in a suitable solvent to obtain 1-(benzyloxy)-3-((E)-3-((E)-prop-1-enyloxy)prop-1-enyl)benzene 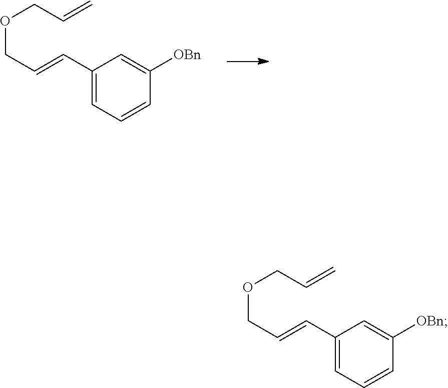 [SMILES: C=CCOC/C=C/c1cccc(OCc2ccccc2)c1 ; C=CCOC/C=C/c1cccc(OCc2ccccc2)c1]
(e) reacting 1-(benzyloxy)-3-((E)-3-((E)-prop-1-enyloxy)prop-1-enyl)benzene with 4-(dimethylamino)-N-((1R,2S)-2-hydroxy-2,3-dihydro-1H-inden-1-yl)picolinamide in the presence of a suitable organometallic catalyst to obtain (2R,3R)-3-(3-(benzyloxy)phenyl)-2-methylpent-4-enal 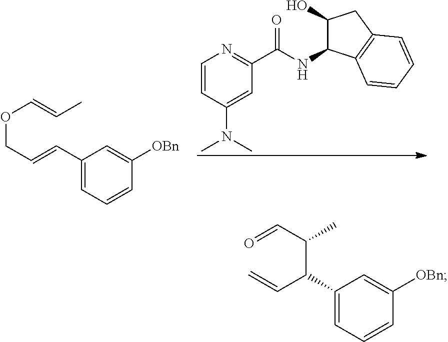
(f) reducing (2R,3R)-3-(3-(benzyloxy)phenyl)-2-methylpent-4-enal in a suitable solvent with a suitable reducing agent to obtain (2R,3R)-3-(3-(benzyloxy)phenyl)-N,N,2-trimethylpent-4-en-1-amine 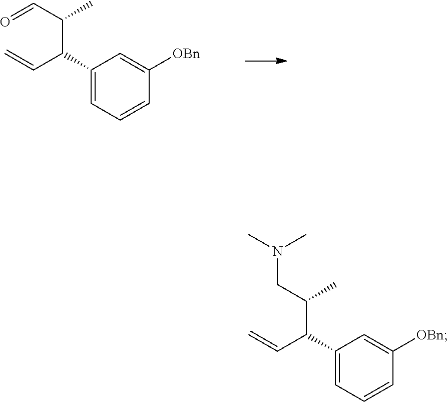
(g) reacting (2R,3R)-3-(3-(benzyloxy)phenyl)-N,N,2-trimethylpent-4-en-1-amine with a suitable acid HX to obtain a salt of (2R,3R)-3-(3-(benzyloxy)phenyl)-N,N,2-trimethylpent-4-en-1-amine 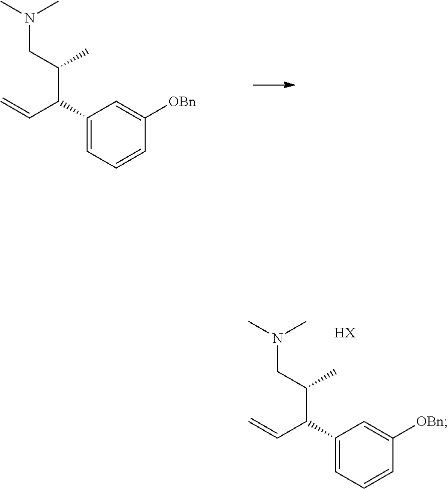
and
(h) hydrogenating the salt of (2R,3R)-3-(3-(benzyloxy)phenyl)-N,N,2-trimethylpent-4-en-1-amine to obtain the salt of tapentadol

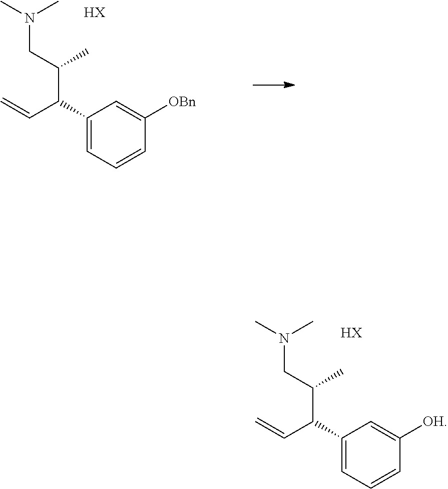

2. The process according to claim 1, wherein the suitable solvent of steps (a) to (g) are each organic solvents, preferably anhydrous organic solvents.

3. The process according to claim 1, wherein the suitable solvent of step (a) is anhydrous dimethylformamide (DMF), anhydrous dimethylsulfoxide (DMSO), anhydrous dimethyl acetamide (DMAc), anhydrous ethanol, anhydrous methanol, anhydrous n-propanol, anhydrous 2-butanol, anhydrous 1-butanol, anhydrous tetrahydrofuran (THF), anhydrous 2-methyltetrahydrofuran (2-MeTHF), anhydrous dioxane, anhydrous toluene, anhydrous ethyl acetate, anhydrous isopropyl acetate, or a mixture thereof.

4. The process according to claim 1, wherein the suitable solvent of step (b) is anhydrous 2-MeTHF, anhydrous THF, anhydrous toluene, anhydrous dioxane, anhydrous methyl tert-buyl ether (MTBE), anhydrous cyclopentyl methyl ether, or anhydrous diethyl ether.

5. The process according to claim 1, wherein step (b) is accomplished using a suitable reducing agent, the reducing agent preferably selected from lithium borohydride, sodium borohydride, lithium aluminum hydride, disobutyl aluminum hydride, or RedAl.

6. The process according to claim 1, wherein the suitable solvent of step (c) is THF, 2-MeTHF, toluene, dioxane, MTBE, cyclopentyl methyl ether, diethyl ether, or a mixture thereof.

7. The process according to claim 1, wherein, the organometallic catalyst of step (d) is [Ir(COD)Cl]₂ or [Ir(COE)₂Cl]₂.

8. The process according to claim 1, wherein the suitable solvent of step (d) is dichloromethane, THF, toluene, dioxane, MTBE, cyclopentyl methyl ether, diethyl ether, acetone, or a mixture thereof.

9. The process according to claim 1, wherein the 4-(dimethylamino)-N-((1R,2S)-2-hydroxy-2,3-dihydro-1H-inden-1-yl)picolinamide is obtained by reacting 4-(dimethylamino)picolinic acid with (1R,2S)-1-amino-2,3-dihydro-1H-inden-2-ol in a suitable solvent, preferably anhydrous DMF

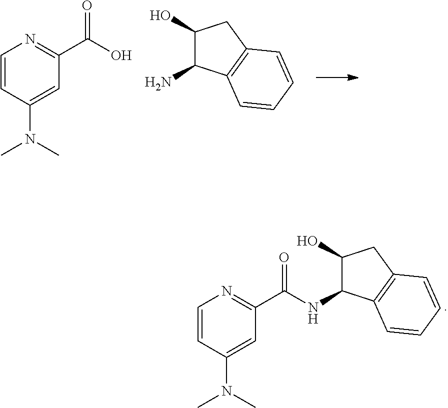 [SMILES: CN(C)c1ccnc(C(=O)O)c1 ; N[C@@H]1c2ccccc2C[C@@H]1O ; CN(C)c1ccnc(C(=O)N[C@@H]2c3ccccc3C[C@@H]2O)c1]

10. The process according to claim 1, wherein the organometallic catalyst of step (e) is [CpRu(MeCN)₃]PF₆ or [Cp*Ru(MeCN)₃]PF₆.

11. The process according to claim 1, wherein the suitable solvent of step (e) is anhydrous THF, anhydrous 2-MeTHF, anhydrous toluene, anhydrous dioxane, anhydrous MTBE, anhydrous cyclopentyl methyl ether, anhydrous diethyl ether, or a mixture thereof.

12. The process according to claim 1, wherein the suitable solvent of step (f) is THF, 2-MeTHF, toluene, dioxane, MTBE, cyclopentyl methyl ether, diethyl ether, dichloromethane, dichloroethane, or a mixture thereof, preferably THF or 2-MeTHF.

13. The process according to claim 1, wherein the suitable solvent of step (g) is MTBE, cyclopentyl methyl ether, diethyl ether, acetonitrile, or a mixture thereof.

14. The process according to claim 1, the suitable acid HX of step (g) is hydrochloric acid, hydrobromic acid, sulfuric acid, methanesulfonic acid, fumaric acid, maleic acid, acetic acid, oxalic acid, succinic acid, malic acid, tartaric acid, mandelic acid, lactic acid, citric acid, glutaminic acid, acetylsalicylic acid, nicotinic acid, aminobenzoic acid, α-lipoic acid, hippuric acid, or aspartic acid, preferably hydrochloric acid.

15. The process according to claim 1, wherein the salt of step (g) is obtained by seeding with crystals of the salt of (2R,3R)-3-(3-(benzyloxy)phenyl)-N,N,2-trimethylpent-4-en-1-amine. Such seeds were obtained the seeds by carefully running the desired crystallization to afford some crystalline solids and these solids were then used to crystallize the bulk material.

16. The process according to claim 1, wherein the hydrogenation of step (h) is accomplished with a hydrogenation catalyst, preferably palladium catalyst on carbon.

17. The process according to claim 1, wherein the salt of tapentadol is converted to tapentadol by reaction with a base.

18. (2R,3R)-3-(3-(benzyloxy)phenyl)-N,N,2-trimethylpent-4-en-1-amine or a salt thereof.

19. A salt of (2R,3R)-3-(3-(benzyloxy)phenyl)-N,N,2-trimethylpent-4-en-1-amine.

20. The chloride salt of (2R, 3R)-3-(3-(benzyloxy)phenyl)-N,N,2-trimethylpent-4-en-1-amine.

21. A process for synthesis of a salt of tapentadol, the process comprising reducing (2R,3R)-3-(3-(benzyloxy)phenyl)-2-methylpent-4-enal in a suitable solvent with a suitable reducing agent to directly obtain tapentadol

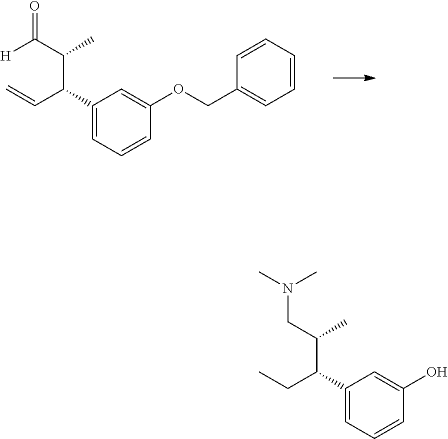 [SMILES: C=C[C@@H](c1cccc(OCc2ccccc2)c1)[C@@H](C)C=O ; CC[C@@H](c1cccc(O)c1)[C@@H](C)CN(C)C]

22. The process according to claim 21, wherein the reduction is accomplished with a hydrogenation catalyst, preferably palladium catalyst on carbon.

23. The process according to claim 21, wherein the suitable solvent is an organic solvent, preferably an anhydrous organic solvent.
